(19) United States
(12) Patent Application Publication
(10) Pub. No.: US 2006/0106387 A1
(43) Pub. Date: May 18, 2006

(54) Spinal plate system and method of use

(75) Inventors: Jonathan Fanger, Fall River, MA (US); Eric D. Kolb, Quincy, MA (US); Stephen Johnson, Johnston, RI (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 10/990,001

(22) Filed: Nov. 16, 2004

(51) Int. Cl. A61F 2/30

(52) U.S. Cl. 606/69

(57) ABSTRACT

A bone plate system is provided in which the system comprises an elongate bone plate having a first surface, a second bone-contacting surface opposed to the first surface, a maximum plate width, a minimum plate width, and a plurality of apertures extending from the first surface through to the second surface for receiving bone screws. The system additionally includes a plurality of bone screws capable of insertion into bone, each screw having a major screw diameter and a minor screw diameter. The dimensions of the bone plate and bone screws being such that the ratio of the maximum plate width to the major screw diameter is less than or equal to approximately 2.7.

### CROSS-REFERENCE TO RELATED APPLICATIONS

Not applicable.

### STATEMENT REGARDING FEDERALLY SPONSORED RESEARCH

Not Applicable.

### FIELD

The present invention relates to fixation devices used in orthopaedic and spinal surgery and particularly to bone fixation plates useful for positioning and immobilizing bone segments.

### BACKGROUND

For a number of known reasons, bone fixation devices are useful for promoting proper healing of injured or damaged vertebral bone segments caused by trauma, tumor growth, or degenerative disc disease. The fixation devices immobilize the injured bone segments to ensure the proper growth of new osseous tissue between the damaged segments. These types of bone fixation devices often include internal bracing and instrumentation to stabilize the spinal column to facilitate the efficient healing of the damaged area without deformity or instability, while minimizing any immobilization and post-operative care of the patient.

One such device is an osteosynthesis plate, more commonly referred to as a bone fixation plate, that can be used to immobilize adjacent skeletal parts such as bones. Typically, the fixation plate is a rigid metal or polymeric plate positioned to span bones or bone segments that require immobilization with respect to one another. The plate is fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed.

Such plates have been used to immobilize a variety of bones, including vertebral bodies of the spine. These bone plate systems usually include a rigid bone plate having a plurality of screw openings. The openings are either holes or slots to allow for freedom of screw movement. The bone plate is placed against the damaged vertebral bodies and bone screws are used to secure the bone plate to the spine, usually with the bone screws being driven into the vertebral bodies. Exemplary systems are described in U.S. Pat. No. 6,159,213 to Rogozinski; U.S. Pat. No. 6,017,345 to Richelsoph; U.S. Pat. No. 5,676,666 to Oxland et al.; U.S. Pat. No. 5,616,144 to Yapp et al.; U.S. Pat. No. 5,549,612 to Yapp et al.; U.S. Pat. No. 5,261,910 to Warden et al.; and U.S. Pat. No. 4,696,290 to Steffee.

Despite the existence of these bone plate systems, there remains a need for a bone plate system that can be implanted with a minimum number of steps and with minimal tissue retraction, tissue dissection, and damage.

### SUMMARY

Disclosed herein are bone plate systems comprising a bone plate and bone screws having unique geometry and dimensions that minimize the width of the plate. In one aspect, the bone plate has a width that is less than that of conventional bone plates, and the bone screws used with the plate have a larger major diameter than conventional bone screws. Combined, these features facilitate bone plate fixation with minimal damage to soft tissue and bone, and also allow implantation of the bone plate system with fewer steps.

The narrow width of the bone plate is advantageous as it reduces trauma to soft tissue by requiring minimal tissue retraction and dissection for implantation. As a result, patients suffer less discomfort and can recover more quickly. In addition, the large diameter of the bone screws provide enhanced fixation such that fewer bone screws are required. For example, in one embodiment, it is possible to implant the plate using only one bone screw per vertebral body. Compared with conventional bone plates, the present system can thus reduce the magnitude of osseous tissue damage incurred by bone structures due to bone screws. As a further advantage, the number of steps required to implant the bone plate system is reduced because the number of screws a surgeon must implant is reduced.

In one embodiment, the bone plate system comprises an elongate bone plate having a first surface, a second bone-contacting surface opposed to the first surface, a maximum plate width, and a plurality of apertures extending through the plate from the first surface to the second surface. The system also includes a plurality of bone screws matable within the apertures for insertion into bone, each screw having a major screw diameter. The dimensions of the bone plate and bone screws are such that, in an exemplary embodiment, the ratio of the maximum plate width to the major screw diameter is less than or equal to approximately 2.7.

In one aspect, the bone plate system includes additional features such as a locking mechanism for preventing bone screw backout. The bone plate can also include bone-engaging protrusions extending from at least one surface of the plate to provide enhanced rotational and torsional stability. These protrusions can extend from a side edge of the bone plate.

In another aspect, the apertures in the bone plate are aligned along a longitudinal axis of the bone plate. In this embodiment, each aperture is adapted to be positioned adjacent a different vertebral body.

In another embodiment, a bone plate has a first surface and an opposed second, bone-contacting surface, and a plurality of apertures extending through the bone plate from the first surface to the second surface, such that each of the plurality of apertures is adapted to receive a bone screw and is aligned with a longitudinal axis of the bone plate. The bone plate further includes an integrated retaining member to inhibit backout of a screw from the apertures. Moreover, the bone plate has a width that varies along the longitudinal axis including a maximum width and a minimum width, wherein the ratio of the maximum width to a minimum diameter of the apertures, measured in a direction transverse to the longitudinal axis, is less than or equal to approximately 2.5.

In a further embodiment, the bone plate system includes a bone plate having a superior end, a central portion, an inferior end, a first surface, and a second, bone-contacting surface opposed to the first surface. The bone plate includes a plurality of apertures extending therethough from the first surface to the second surface. The system also includes a plurality of bone screws implantable within the apertures for insertion into bone, each screw having a major screw diameter. The bone plate also includes a width which may vary along a portion of its length.

In yet another aspect, the bone plate system includes a bone plate having a first surface and a second bone-contacting surface opposed to the first surface, and a plurality of apertures extending from the first surface to the second surface for receiving bone screws. The apertures can be positioned along a longitudinal axis of the bone plate and spaced such that each aperture is adapted for placement adjacent to a different vertebral body. The bone plate can have an elongate shape with a width that varies along the longitudinal axis of the bone plate, wherein a maximum width is less than or equal to approximately 10.5 mm. The system also includes a plurality of bone screws capable of insertion through an aperture into bone, wherein each screw has a major screw diameter of at least approximately 4.6 mm.

In an additional aspect, the bone plate can include a portion with a greater width than other portions of the bone plate. The portion of greater width may have two apertures oriented transverse to a longitudinal axis of the bone plate and adapted for placement adjacent the same vertebral body. In one embodiment, the bone plate also includes at least one additional aperture at a portion of the plate having a lesser width and the ratio of the bone plate width at the portion of the plate having a lesser width, measured across the at least one additional aperture, to the major bone screw diameter is equal to or less than approximately 2.5.

In yet another aspect, the bone plate includes a portion having a wider width at one end of the bone plate. The wider end of the plate preferably includes multiple apertures arranged across the wider width, the multiple apertures being oriented transverse to a longitudinal axis of the plate and each aperture being adapted for placement adjacent to the same vertebral body. The end of the plate having a lesser width preferably includes at least one aperture, each aperture in the end having a lesser width is aligned with the longitudinal axis of the plate and is adapted for placement adjacent to a different vertebral body. In one embodiment, the ratio of the plate width as measured across an aperture in the narrow end to the major screw diameter is less than approximately 2.7.

The present invention also encompasses methods of implanting a bone plate system. In one embodiment, the method includes providing an elongate bone plate having a first surface, a second bone-contacting surface opposed to the first surface, a maximum plate width, a minimum plate width, and a plurality of apertures extending therethrough from the first to the second surface for receiving bone screws. A plurality of bone screws are also provided, each screw having a major screw diameter and a minor screw diameter, wherein the ratio of the maximum plate width to the major screw diameter is less than or equal to approximately 2.7. The method further includes the steps of creating at least one incision to provide access to a site on or adjacent to a patient's spinal column, inserting the bone plate through the at least one incision, and placing the bone plate at a desired location spanning at least two vertebral bodies. The bone screws are then inserted through the at least one incision and through an aperture in the bone plate. In one exemplary technique, each bone screw is implanted in a different vertebral body.

### BRIEF DESCRIPTION OF THE DRAWINGS

The invention can be more fully understood from the following detailed description taken in conjunction with the accompanying drawings, in which:

FIG. 1 is a perspective view of an exemplary embodiment of the present bone plate system including a bone plate and bone screws;

FIG. 2A is a top view of another embodiment of an exemplary bone plate;

FIG. 2B is a side view of the bone plate of FIG. 2A;

FIG. 2C is an end view of the bone plate of FIG. 2A;

FIG. 2D is a side view of an alternative embodiment of a bone plate;

FIG. 2E is a sectional view of the plate shown in FIG. 2D along lines 2E-2E;

FIG. 3A is a perspective view of one embodiment of a bone screw useful with the present bone plate system;

FIG. 3B is a side sectional view of the bone screw of FIG. 3A;

FIG. 4A is a top view of another embodiment of an exemplary bone plate;

FIG. 4B is a side view of the bone plate of FIG. 4A;

FIG. 5 is a top view of another embodiment of an exemplary bone plate;

FIG. 6 is a top view of a further embodiment of an exemplary bone plate;

FIG. 7 is a top view of an additional embodiment of an exemplary bone plate;

FIG. 8A is a perspective view of a bone plate with bone-engaging spikes;

FIG. 8B is a side view of the bone plate of FIG. 8A along lines 8B-8B of FIG. 8A;

FIG. 9A is a top view of yet another embodiment of a bone plate with bone-engaging spikes;

FIG. 9B is an end view of the bone plate of FIG. 9A;

FIG. 10A is a partial view of an exemplary locking mechanism for a bone plate, illustrating the locking mechanism in an unlocked position;

FIG. 10B is a view of the locking mechanism of FIG. 10A after rotating the locking mechanism into a locked position; and

FIG. 11 is a perspective view of an implanted bone plate system on a patient's spinal column, illustrating the plate attached to the anterior surfaces of adjacent vertebrae of the cervical spine.

### DETAILED DESCRIPTION

In general, disclosed herein are spinal fixation plates having at least two apertures for receiving a bone screw. The plate may be adapted to be attached to adjacent vertebrae to maintain the vertebrae in a fixed position and thereby provide biomechanical stability to the vertebrae. The shape and structure of the bone plate system facilitate its use in a variety of surgical procedures, including minimally invasive surgical procedures. In one aspect, the plate has a width that is narrower than conventionally used bone plates, permitting the use of a smaller incision. In addition, the bone screws used with the plate system have a larger major diameter than conventional bone screws. As a result, fewer bone screws are needed to affix the plate to bone, and it is possible to use only a single bone screw for each vertebral body to which the plate is to be affixed, thus reducing the time and number of steps required by a surgeon to implant the plate.

The following exemplary embodiments are described herein with reference to bone plates used to span and immobilize adjacent vertebral bodies in spinal fixation techniques. However, it is understood that the bone plate systems described herein may be applicable to the fixation of any type of adjacent bones or bone segments.

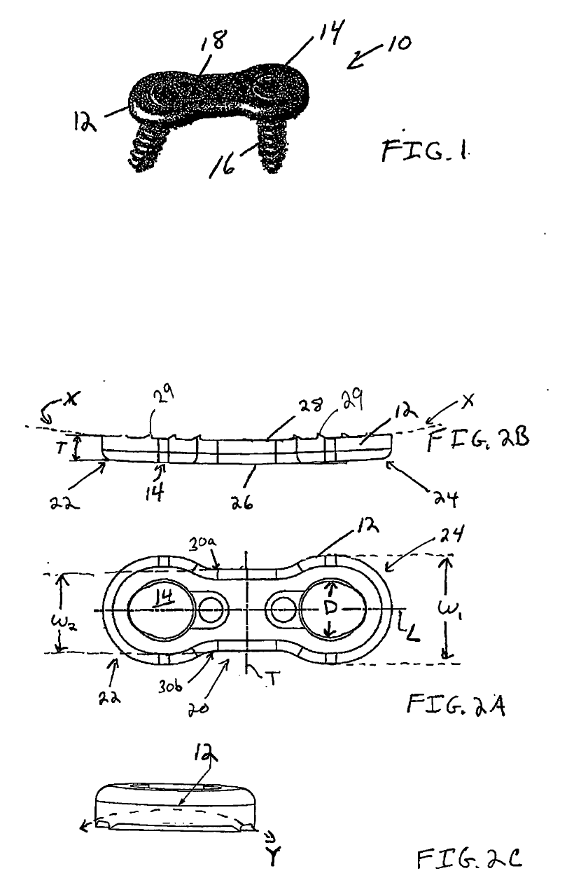

FIG. 1 illustrates one embodiment of the bone plate system 10, which includes a bone plate 12 having apertures 14 adapted for receiving bone screws 16. The apertures are positioned along the longitudinal axis L (FIG. 2A) of the bone plate and spaced for fixing separate bone segments such that only a single bone screw is used to secure the plate per vertebral body. The bone plate may also include a locking mechanism 18 to provide bone screw backout resistance. Although the system illustrated in FIG. 1 is used as a one level plate (i.e., bridging two vertebral bodies), as described in more detail below, the system is applicable to plates that bridge more than two vertebral bodies 60, as for example, illustrated in FIG. 11.

FIGS. 2A, 2B, and 2C illustrate a top, side, and end view of bone plate 12, respectively. As shown, the bone plate has a generally elongate shape with a mid-portion 20 positioned between an inferior end 22 and a superior end 24, and a longitudinal axis L extends between the inferior and superior ends 22, 24. The bone plate 12 further includes a non-bone-contacting surface 26 and a bone-contacting surface 28, as well as opposed lateral sides 30a, 30b extending therealong between the inferior and superior ends 22, 24. The plate may also include bend zones (not shown), which are thinner areas of the plate that enable a surgeon to bend a plate to match the contour of an anatomical structure on which the plate will be mounted.

As indicated above, plate 12 is adapted to mate to at least two vertebrae. The system and plate illustrated in FIGS. 1-2E is adapted to mate between superior and inferior vertebrae, with the inferior end 22 being affixed to an inferior vertebra and superior end 24 affixed to a superior vertebra. Accordingly, the plate 12 preferably includes one or more apertures 14 for receiving a fastening element, such as a bone screw 16, to attach the plate to adjacent vertebrae. Each aperture 14 extends through the plate 12 from a non-bone-contacting surface 26 to a bone-contacting surface 28. In one embodiment, the bone plate is adapted for fixation to the spine using only one bone screw per vertebral body. In this embodiment, the apertures are arranged along the longitudinal axis L of the bone plate. FIGS. 1-2C illustrate a bone plate in which the plate 12 will span two vertebral bodies and each aperture 14 corresponds to the desired position of a bone screw in a vertebral body. The bone plate of FIGS. 2D and 2E spans three vertebral bodies and each aperture 14 corresponds to the desired position of a bone screw in a vertebral body.

Bone plate 12 can have a variety to shapes, however, the bone plate generally includes a non-uniform width along its length. In one embodiment, the bone plate has a substantially hourglass shape as illustrated in FIGS. 1 and 2A. As shown, the plate 12 has a maximum (or widest) width at the inferior end 22 and superior end 24 and a minimum (or narrowest) width at mid-portion 20. The maximum width of bone plate 12, illustrated as W₁ in FIG. 2A, is preferably less than approximately 14 mm, more preferably less than approximately 12 mm, and even more preferably less than or equal to approximately 10.5 mm. In yet another embodiment, the maximum width of the bone plate is in the range of approximately 5 mm to approximately 10.5 mm. The minimum width, shown as W₂ in FIG. 2A, is generally located across at the mid-portion 20 of bone plate 12 and is preferably in the range of approximately 2 mm to approximately 10 mm, and more preferably from approximately 4 mm to approximately 10 mm. The position of maximum width W₁ usually corresponds to the region of plate that has an aperture while the minimum width W₂ generally corresponds to a region of plate between apertures. One skilled in the art will appreciate that a variety of other bone plate shapes that include a maximum and minimum width can be substituted for the hourglass shape illustrated herein. Plates having a uniform width are also encompassed.

Apertures 14 in bone plate 12 can have a variety of shapes, as long as they are suitable to receive a fixation element. In one exemplary embodiment, the apertures 14 can be generally circular. In another embodiment, including the illustrated embodiment, the apertures can be in the form of a circle that is slightly elongated in the longitudinal direction. One or more apertures 14 include a diameter that may vary between the non-bone-contacting surface 26 and the bone-contacting surface 28. In one exemplary embodiment, one or more of the apertures 14 has a diameter that tapers from the non-bone contacting surface 26 to the bone-contacting surface 28. For example, as shown in FIGS. 2D and 2E, the apertures of the exemplary plate have a minimum diameter D adjacent the bone-contacting surface 28, measured transverse to the longitudinal axis of the plate. One skilled in the art will appreciate that the aperture diameter D depends on the bone screw diameter and the optional use of washers or locking mechanisms. In one exemplary embodiment, the aperture diameter D is in the range of approximately 4 mm to approximately 6 mm. In another embodiment, the aperture diameter D is greater than or equal to approximately 4.6 mm, and in yet another embodiment the aperture diameter D is greater than or equal to approximately 5.2 mm. As discussed further below, the bone plate width and bone screw diameters are matched to one another and in one embodiment, the ratio of the maximum plate width W₁ to the aperture diameter D is preferably less than or equal to approximately 2.7. In another embodiment, the ratio of maximum plate width to the aperture minimum diameter D is preferably in the range of approximately 1.1 to approximately 2.7, more preferably it is in the range of approximately 1.5 to approximately 2.5, and even more preferably it is in the range of approximately 1.9 to approximately 2.5. In an exemplary embodiment, the range is approximately 2.0 to approximately 2.3. In another embodiment the ratio of the maximum plate width W₁ to the aperture minimum diameter D is less than or equal to approximately 2.5

The spacing between apertures 14 depends on distance between vertebral bodies (and/or bone grafts) along the length of a patient's spinal column. In one embodiment, where the bone plate is adapted to provide a single screw per vertebral body (or bone graft), the exemplary hole-to-hole spacing between apertures is preferably in the range of approximately 8 mm to approximately 25 mm. In one embodiment, the ratio of plate width to hole-to-hole spacing is preferably in the range of approximately 0.2 to approximately 1.0, and even more preferably in the range of approximately 0.3 to approximately 0.9.

Bone plate 12 also includes a bone plate thickness T (FIG. 2B) between non-bone-contacting surface 26 and bone-contacting surface 28. In general, the bone plate thickness is in the range of approximately 1 mm to approximately 3 mm. For the purpose of measuring bone plate thickness, plate surface features are preferably ignored.

In an exemplary embodiment, the plate 12 is adapted to be mounted upon the anterior surface of vertebrae in the cervical or lumbar region of the spine. For example, the bone-contacting surface 28 of the exemplary plate 12 can have a longitudinal curve X that approximates the lordotic curvature of the vertebrae upon which the plate is to be mounted. As shown in FIG. 2B, the exemplary plate 12 has a longitudinal curve X that extends in the sagital plane (i.e., in the superior-inferior direction) and has a constant radius along the length of the plate 12. Alternatively, the plate 12 may comprise a plurality of longitudinal segments that are configured to collectively provide the plate with a longitudinal curvature that approximates the lordotic curvature of the vertebrae. For example, one or more of the longitudinal segments may have a longitudinal curvature or may be oriented at angle relative to the other longitudinal segments.

While the exemplary plate 12 may be curved only along longitudinal axis L, in another embodiment, plate 12 can also include a transverse curve Y that approximates the transverse curvature of the vertebrae upon which the plate is to be mounted, as illustrated in FIG. 2C. The plate 12 may have a transverse curvature along the length of the plate 12 or along discrete longitudinal segments of the plate. For example, the mid-portion 20 of the exemplary plate 12 may have a transverse curvature that approximates the transverse curvature of the vertebrae. Alternatively, the inferior end 22 and/or the superior end 24 may have a transverse curvature.

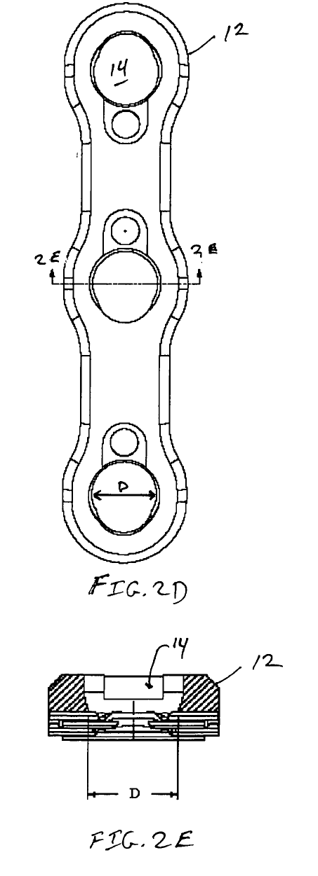

The bone screws 16 useful with the present system preferably have a larger diameter than conventional bone screws used for fixing a bone plate to a vertebral segment. FIGS. 3A and 3B illustrate one embodiment of bone screw 16 for use with the bone plate system. Bone screw 16 preferably includes an elongate body 30 with threads 32 along at least a portion thereof, and a head 34 for mating with bone plate 12 and for optionally receiving a driver tool. The bone screw 16 has a major screw diameter SD₁ and a minor screw diameter SD₂.

In one embodiment, the dimensions of the bone plate and the bone screws are adapted such that the ratio of the maximum plate width to the major screw diameter is less than or equal to approximately 2.7. In another embodiment, maximum plate width to the major screw diameter is preferably in the range of approximately 1.1 to approximately 2.7, more preferably in the range of approximately 1.5 to approximately 2.5, and even more preferably in the range of approximately 1.9 to approximately 2.5. In an exemplary embodiment, the range is approximately 2.0 to approximately 2.3.

In one aspect of the present system, the major diameter of bone screw 16 is larger than the major diameter of conventional bone screws. The major screw diameter SD₁ for a standard screw used with system 10 is in the range of approximately 4.4 to approximately 5.0 mm. In an exemplary embodiment, the major screw diameter of a standard screw is equal to or more than approximately 4.6 mm. In another embodiment, system 10 can include oversized revision screws, which are particularly useful when a problem is encountered with implanting the screws (e.g., a new oversized hole must be drilled in place of an existing hole in a vertebral body). In one embodiment, the oversized revision screws have a major diameter in the range of approximately 5.0 mm to approximately 5.6 mm. In an exemplary embodiment, the oversized revision screws have a major screw diameter of approximately 5.2 mm. In yet another aspect, the major diameter of the revision screw is at least 0.6 mm greater than the major diameter of the standard screw.

The bone plate system may include different types of bone screws having varying functionalities. For example, the bone screws can be of a rigid type in which after a screw locking mechanism is engaged, movement of the screw in any direction is prevented. The bone screws can also be of a semi-rigid type in which after a screw locking mechanism is engaged, screw backout is prevented, but the screw is able to move in all directions (i.e., polyaxially). Further, the bone screws can also be of a hybrid type in which after a screw locking mechanism is engaged, screw backout is prevented, but the screw is able to move in only one selected direction (e.g., the superior-inferior or the transverse direction). Moreover, the bone screws may translate within an aperture of a plate. For example, a bone screw may translate along the length of an elongated slot defining an aperture in the plate. One skilled in the art will appreciate that a bone plate system may be provided having any single screw type or a combination of all or any of the screw types.

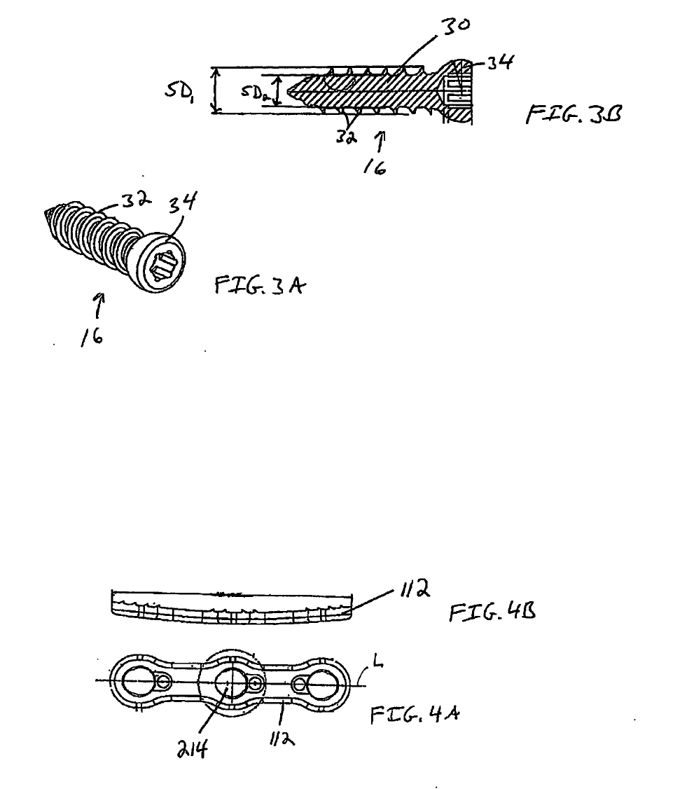

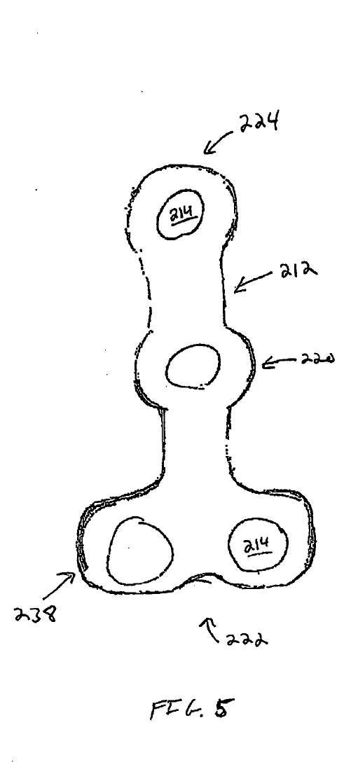

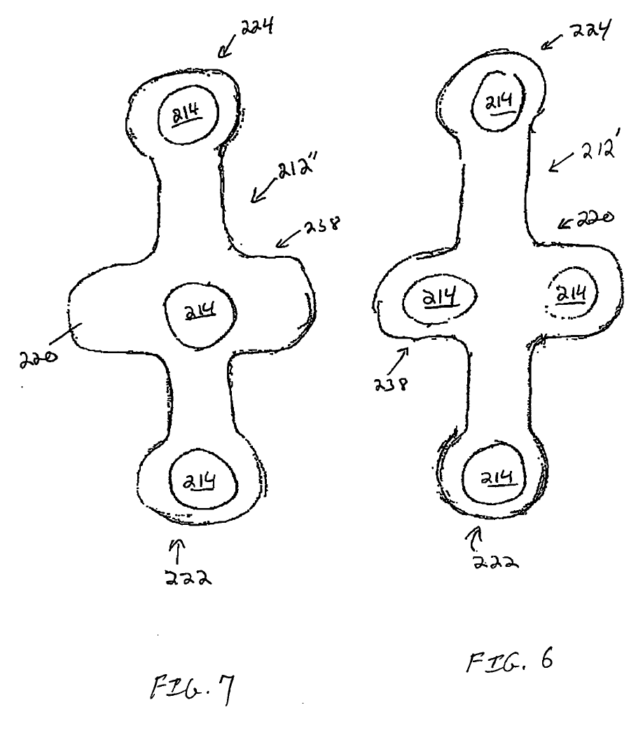

As noted above, the present bone plate system also encompasses multi-level plates, such as a two level plate 112 shown in FIGS. 4A and 4B. Multi-level plates can span more than two adjacent vertebrae, for example, the illustrated plate 112 can span three vertebral bodies. The illustrated multi-level plate includes three apertures 214, with only a single aperture adjacent each vertebral body. In another embodiment, the multi-level plate can be a three or more level plate having four or more apertures for receiving bone screws. The multi-level plates can also be useful for spanning and anchoring bone grafts. For example, the center aperture 214 of bone plate 112 can receive a bone screw for fixing the plate to a bone graft. As noted above, in this embodiment, each aperture 214 is preferably adapted to be the only aperture positioned adjacent to a different vertebral body or a bone graft.

The multi-level plates may include the features of the single level bone plates described above, and thus the dimensions and geometry of the multi-level plates are similarly adapted for a variety of surgical procedures, including minimally invasive surgical procedures. That is, the ratios and dimensions discussed above with respect to a single level plate are equally applicable to multi-level plates.

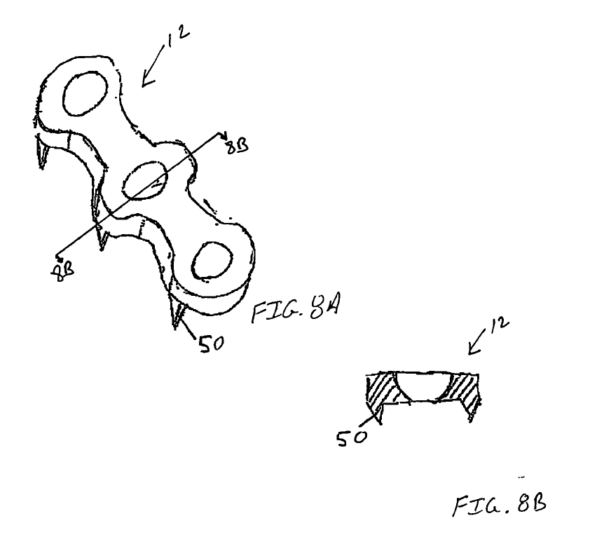

The present system also encompasses plate system designs in which only a portion of the plate has the dimensions and geometry discussed above. That is, only a portion of the plate is designed such that only a single aperture will be placed adjacent a vertebral body while another portion of the plate can have more traditional or alternative designs. In one embodiment, the bone plate can include an extra wide portion that includes multiple screw apertures adapted to be positioned adjacent to a single vertebral body. Examples of such designs are illustrated in FIGS. 5 and 6.

The plates illustrated in FIG. 5 include an extra wide portion 238 at one end thereof. As shown, the extra wide portion includes multiple apertures 214 adapted for positioning adjacent to a single vertebral body. In use, multiple bone screws can be used to fix the wider end 222 of the bone plate (FIG. 5) to a single bone segment. A person skilled in the art will appreciate that an extra wide portion 238 at the inferior or superior end of the bone plate can have a variety of shapes. In addition, an extra wide end of the bone plate 212 can be adapted for mating with a drill guide.

FIGS. 6 and 7 illustrates a plate 212′, 212″ having an extra wide portion 238 at the mid-portion 220. This extra wide portion 238 can provide additional surface area for fixation to a vertebra. The extra wide portion can provide additional stability to the bone graft and helps to prevent shifting under load, and it can have a single aperture (FIG. 7) or more than one aperture (FIG. 6).

The bone plate 212, 212′, 212″ illustrated in FIGS. 5-7, also includes a region that is adapted for fixing a single bone screw per vertebral body thereby presenting a minimal profile in this region of the plate. This region has the dimensions and geometry discussed above with respect to the single level plate. For example, the superior end 224 of plates 212, 212′, and 212″ can include the uniquely matched plate width and major screw diameter. The major width W₁ used to determine the ratio of plate width to major screw diameter is measured across an aperture in a portion of the bone plate adapted for fixing a single bone screw per vertebral body. For example, the plate width in FIGS. 5 and 6 can be measured at the superior end 224 or the inferior end 222 across the aperture 214 in the superior or inferior end.

In an alternative embodiment, illustrated in FIG. 7, the plate 212″ has an extra wide central portion 220 that includes only a single aperture adapted for positioning adjacent to a vertebral body. Preferably, the inferior and superior ends of the plate 212″ have a minimal profile with the dimensions and geometry discussed above with respect to the single level plate. The maximum plate width as measured at inferior end 222 and/or at superior end 224 preferably falls within the desired bone plate system ratios. In particular, ratio of the maximum plate width measured across an aperture at the inferior end (and/or the superior end) to the major screw diameter is preferably less than or equal to approximately 2.7. In another embodiment, the maximum plate width measured across an aperture at the inferior end (and/or the superior end) to the major screw diameter is in the range of approximately 1.1 to approximately 2.7, more preferably in the range of approximately 1.5 to approximately 2.5, and even more preferably in the range of approximately 1.9 to approximately 2.5. In an exemplary embodiment, the range is approximately 2.0 to approximately 2.3.

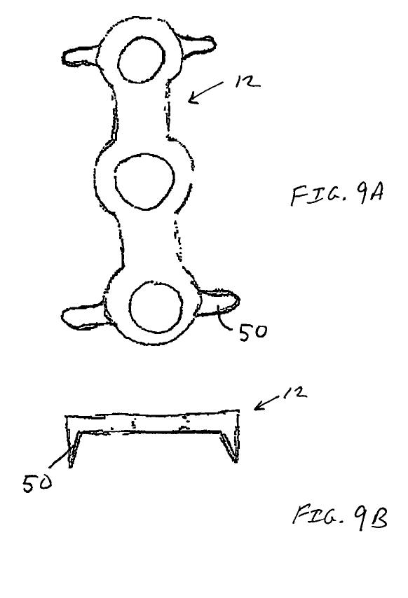

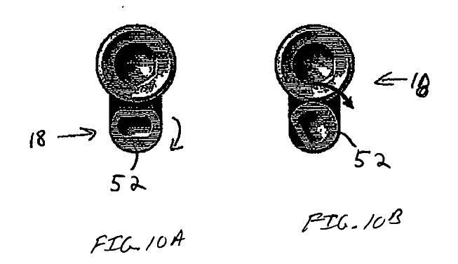

The various embodiments of the bone plate system can include additional features that provide bone plate stabilization. For example, the bone contacting surface of the bone plate may include surface features that facilitate engagement of the plate to a surface of a vertebra. In the exemplary plate 12 described above, for example, the bone-contacting surface 28 of the plate 12 may include one or more cleats 29 to facilitate engagement of the bone contacting surface 28 to a surface of a vertebra. The cleats 29, in the exemplary embodiment, are oriented transverse to the longitudinal axis L of the plate 12 and span the width of the plate 12. The bone plates may include other surface features. For example, in one embodiment, the bone plate (12, 112, 212) includes one or more bone-engaging spikes positioned adjacent to the lateral edge of the bone plate. As shown in FIGS. 8A and 8B, the three pairs of bone-engaging spikes 50 extend downward from the bone plate to minimize rotational or torsional plate movement. In another embodiment, bone-engaging spikes 50 can be spaced from the lateral edge of the bone plate as shown in FIGS. 9A and 9B. As shown, a pair of bone-engaging spikes 50 extend outward from the side of the bone plate and then extend downward. The spacing between bone-engaging spikes in a pair may correspond to the approximate width of a vertebral body such that the spikes are adapted to be positioned on the sides of a vertebral body to promote rotational and/or torsional stability.

One skilled in the art will appreciate that the bone-engaging spikes 50 can be positioned anywhere along the edge of the bone plate, or elsewhere on the bone-contacting surface of the plate. In one embodiment, the bone-engaging spikes are positioned on the edge of the bone plate at the maximum plate width. In another embodiment, at least one set of bone-engaging spikes is positioned adjacent an aperture, and in yet another embodiment, bone-engaging spikes are positioned adjacent to each aperture.

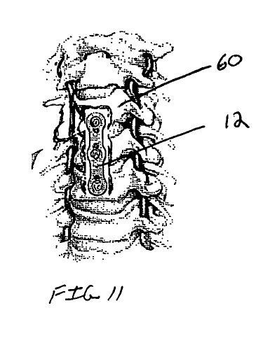

The various embodiments of the bone plate system can additionally include a locking or retaining mechanism 18 for preventing bone screw backout. For example, the bone plate (12, 112, 212) can include an integrated locking mechanism present on the non-bone-contacting surface of the plate 12. The integrated locking mechanism can be in the form of a rotatable cam 52 which can be rotated between a locked and an unlocked position. FIGS. 10A and 10B illustrate a cut-away view of cam 52 in an unlocked (FIG. 10A) and locked position (FIG. 10B). In the locked position, the rotatable cam 52 is forced against the head of the bone screw to provide bone screw backout resistance. An exemplary cam-type locking mechanism is described in U.S. Pat. No. 5,549,612, which is incorporated by reference herein in its entirety. One skilled in the art will appreciate that a variety of other retaining mechanisms can be used as well, including those that are integrated with the plate and those that are not. Exemplary retaining mechanisms include locking washers, locking screws, and bone screw covers. One skilled in the art will appreciate that various combinations of locking mechanisms can be used as well.

The exemplary bone plate systems described herein may be constructed of any biocompatible material including, for example, metals, such as stainless steel and titanium, polymers, and composites thereof. In certain exemplary embodiments, the bone plate system may be constructed of a bio-resorbable material, such as, for example polylactic acid (PLA) and polyglycolic acid (PGA), and blends or copolymers thereof.

The bone plate system, as described above, can be implanted by any type of surgical procedure, including minimally invasive surgical techniques. For example, the exemplary bone plate systems described herein may be implanted through a minimally invasive access system, including for example a port or a retractor. Exemplary minimally invasive access systems and methods are described in U.S. Pat. No. 6,159,179; U.S. Patent Application Publication No. 2003/0083689; U.S. Patent Application Publication No. 2003/0083688; and U.S. Patent Application Ser. No. 60/589,727, filed Jul. 21, 2004, each of which is incorporated herein by reference. One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A bone plate system, comprising:
an elongate bone plate having a first surface, a second bone-contacting surface opposed to the first surface, a maximum plate width, and a plurality of apertures extending from the first surface to the second surface for receiving bone screws, the second bone-contacting surface having a sagital curvature approximate to a lordotic curvature of an anterior surface of vertebrae upon which the plate is adapted to be mounted; and
a plurality of bone screws capable of insertion into bone, the screws having a major screw diameter;
wherein the ratio of the maximum plate width to the major screw diameter is less than or equal to approximately 2.7.

2. The system of claim 1, wherein the maximum plate width to the major screw diameter is in the range of approximately 1.1 to approximately 2.7.

3. The system of claim 1, wherein the maximum plate width to the major screw diameter is in the range of approximately 1.5 to approximately 2.5.

4. The system of claim 1, wherein the maximum plate width to the major screw diameter is in the range of approximately 1.9 to approximately 2.5.

5. The system of claim 1, wherein the maximum plate width to the major screw diameter is in the range of approximately 2.0 to approximately 2.3.

6. The system of claim 1, wherein the plurality of apertures are aligned along a longitudinal axis of the bone plate.

7. The system of claim 1, wherein the bone plate has a width at a first end thereof that is greater than the width at a second end thereof.

8. The system of claim 7, wherein the bone plate further includes at least one aperture disposed intermediate the first and second end that is positioned along a longitudinal axis of the bone plate.

9. The system of claim 7, wherein the first end of the bone plate has a plurality of transverse apertures oriented substantially transverse to a longitudinal axis of the bone plate, wherein each of the plurality of transverse apertures is adapted for placement adjacent to the same vertebral body, and wherein the apertures at the second end of the bone plate are aligned along the longitudinal axis of the bone plate and each aperture at the second end of the bone plate is adapted for placement adjacent to a different vertebral body.

10. The system of claim 9, wherein the maximum plate width is measured across an aperture in the second end of the bone plate.

11. The system of claim 1, further including a retaining mechanism for preventing bone screw backout.

12. The system of claim 1, wherein the ratio of the maximum plate width to a maximum plate thickness is in the range of approximately 3 to approximately 8.

13. The system of claim 1, wherein the bone plate further comprises a plurality of bone-engaging protrusions extending from at least one surface thereof.

14. The system of claim 1, wherein each of the plurality of apertures is adapted to be positioned adjacent to a different vertebral body.

15. A bone plate, comprising:
a first surface and an opposed second, bone-contacting surface, a plurality of apertures extending through the bone plate from the first surface to the second surface, each of the plurality of apertures being adapted to receive a bone screw and being aligned with a longitudinal axis of the bone plate, and an integrated retaining member to inhibit backout of a screw from the apertures, the bone plate having a width that varies along the longitudinal axis including a maximum width and a minimum width, wherein the ratio of the maximum width to a minimum diameter of the apertures, measured in a direction transverse to the longitudinal axis, is less than or equal to approximately 2.5.

16. The bone plate of claim 15, wherein the integrated retaining member is a cam positioned on the first surface of the bone plate proximate an aperture.

17. A bone plate system, comprising:
a bone plate having a first surface and a second bone-contacting surface opposed to the first surface, and a plurality of apertures extending from the first surface to the second surface for receiving bone screws, the apertures positioned along a longitudinal axis of the bone plate and spaced such that each aperture is adapted for placement adjacent to a different vertebral body, the bone plate having an elongate shape with a width that varies along the longitudinal axis of the bone plate, wherein a maximum width is less than or equal to approximately 10.5 mm; and
a plurality of bone screws capable of insertion into bone, each screw having a major screw diameter of at least approximately 4.6 mm.

18. The system of claim 17, including an oversized screw having a major diameter of at least approximately 0.6 mm greater than the major screw diameter of the plurality of bone screws.

19. A bone plate, comprising:
an elongate member having a superior end, a inferior end, a first surface, a second bone-contacting surface opposed to the first surface, and a plurality of apertures extending through the elongate member from the first surface to the second surface, the elongate member further including a width at a first end that is greater than a width at a second end, wherein each aperture at the first end is aligned transversely to a longitudinal axis of the elongate member and is adapted to be positioned adjacent to a single vertebral body and each aperture at the second end of the elongate member is aligned with the longitudinal axis and adapted to be positioned adjacent to a different vertebral body; and
wherein the ratio of the maximum bone plate width measured at the second end to a minimum diameter of the apertures, measured in a direction transverse to the longitudinal axis, is less than or equal to approximately 2.5.

20. A bone plate, comprising:
an elongate member having a first end, a central portion, a second end, a first surface, a second bone-contacting surface opposed to the first surface, and a plurality of apertures extending through the elongate member from the first surface to the second surface, the elongate member further including a width in the central portion that is greater than a width at each of the first end and the second ends, wherein the aperture in the first and the aperture in the second end are aligned along a longitudinal axis of the elongate member and is adapted to be positioned adjacent to a different vertebral body; and
wherein the ratio of the maximum bone plate width measured at the first end to a minimum diameter of the apertures, measured in a direction transverse to the longitudinal axis, is less than or equal to approximately 2.5.

21. The bone plate of claim 19, wherein the ratio of the maximum bone plate width measured at the second end to the diameter of the apertures is less than or equal to approximately 2.5.

22. The bone plate of claim 19, wherein the central portion includes at least one aperture positioned along the central longitudinal axis of the elongate member.

23. A method of implanting a bone plate system, comprising:
providing an elongate bone plate having a first surface, a second bone-contacting surface opposed to the first surface, a maximum plate width, a minimum plate width, and a plurality of apertures extending from the first surface through to the second surface for receiving bone screws, the bone plate further including a locking mechanism for preventing bone screw backout,
providing a plurality of bone screws capable of insertion into bone, the screws having a major screw diameter, wherein the ratio of the maximum plate width to the major screw diameter is less than or equal to approximately 2.7;
creating at least one incision to provide access to a site on or adjacent to a patient's spinal column;
inserting the bone plate through the at least one incision and placing the bone plate at a desired location spanning at least two vertebral bodies; and
inserting a bone screw through the at least one incision and through an aperture in the bone plate such that each bone screw is implanted in a different vertebral body and each vertebral body receives only one bone screw.

24. The method of claim 23, wherein the length of each of the at least one incision is equal to or less than the maximum plate width.

25. A bone plate system, comprising:
an elongate bone plate having a first surface, a second bone-contacting surface opposed to the first surface, and a plurality of apertures extending from the first surface to the second surface for receiving bone screws, the second bone-contacting surface having a curvature approximate to a lordotic curvature of an anterior surface of vertebrae upon which the plate is adapted to be mounted;
a plurality of bone screws capable of insertion into bone, the screws having a major screw diameter; and
an oversized screw having a major diameter of at least approximately 0.6 mm greater than the major screw diameter of the plurality of bone screws.

26. The bone plate system of claim 25, further comprising an integrated retaining member to inhibit backout of a screw from an aperture.

27. The bone plate system of claim 25, wherein each aperture of the plurality of apertures is aligned along a longitudinal axis of the plate.
